(19) United States
(12) Patent Application Publication
(10) Pub. No.: US 2013/0210675 A1
(43) Pub. Date: Aug. 15, 2013

(54) COMPOSITIONS AND METHODS FOR CONTROLLED RELEASE OF BIOMOLECULES

(71) Applicants: Su Guo, Beijing (CN); Guoqing Wang, Beijing (CN); Guohao Zhang, Beijing (CN); Can Wang, Beijing (CN); Bei Han, Beijing (CN); Miao Liu, Beijing (CN); Jinxiu Zhang, Beijing (CN); Tao Deng, Beijing (CN); Wanli Xing, Beijing (CN); Jing Cheng, Beijing (CN)

(72) Inventors: Su Guo, Beijing (CN); Guoqing Wang, Beijing (CN); Guohao Zhang, Beijing (CN); Can Wang, Beijing (CN); Bei Han, Beijing (CN); Miao Liu, Beijing (CN); Jinxiu Zhang, Beijing (CN); Tao Deng, Beijing (CN); Wanli Xing, Beijing (CN); Jing Cheng, Beijing (CN)

(73) Assignee: CapitalBio Corporation, Beijing (CN)

(21) Appl. No.: 13/811,136

(22) PCT Filed: Jul. 13, 2011
(86) PCT No.: PCT/CN11/01159

§ 371 Date: Mar. 28, 2013

(30) Foreign Application Priority Data
Jul. 19, 2010 (CN) 201010233646.6

(51) Int. Cl. C12Q 1/68; C08G 69/10; C08B 37/00; C08B 37/08; C07H 21/04

(52) U.S. Cl. CPC C12Q 1/6853; C08B 37/003; C07H 21/04; C08B 37/0039; C08G 69/10
USPC 506/16; 536/23.1; 525/54.2

(57) ABSTRACT

Provided are compositions for controlled release of biomolecules, which comprise conjugates of polymers and biomolecules conjugated through non-covalent interactions. Also provided are methods for controlled release of biomolecules and their use in biochips.

### TECHNICAL FIELD

The invention relates to compositions and methods for controlled release of biomolecules and their uses in biochips.

### BACKGROUND ART

Biochips are increasingly being used in the area of gene detections, showing the advantages in fast analysis, low sample consumption, and high integration. They also have promising uses in disease diagnosis, drug screening, and the medicolegal field.

Especially in the miniaturized biochips for gene amplification, the reaction efficiency is essentially determined by the effective release of the biomolecules. Currently, there are two main strategies of releasing biomolecules in biochip technology. One is direct releasing, in which the samples are well mixed before the reaction and there are no additional releasing steps during the reaction. The other is added-releasing, in which the samples are added into the reaction system in sequence by a flow. In the direct releasing model, it is impossible to achieve multiple amplifications separately. Further, additional structures, such as parallel channels, are required to control the flow of separate samples. These can lead to more complicated chip design and higher fabrication cost. Some researchers developed added-releasing strategies, in which reagents were deposited into the biochips before the reaction buffers were added to initiate amplification reactions. But there are also some disadvantages in the pre-deposited biochips. If the reagents were deposited into the biochips covalently, the reaction efficiency could be reduced because of reduction of the biomolecules' activities. Otherwise, if the reagents were deposited into the chips by a non-covalent interaction, contamination and loss could occur during the following sample adding step. So an efficient, simple and controllable release method is needed to improve the reaction efficiency of biochips.

### SUMMARY OF THE INVENTION

The present invention relates to a controlled release conjugate comprising a biomolecule conjugated with a polymer, and its methods of use. Therefore, in one aspect, provided herein is a controlled release conjugate comprising a biomolecule conjugated with a polymer through a non-covalent bond, which releases the biomolecule from the polymer by a physical treatment and/or change in an environmental condition.

In some embodiments, the non-covalent bond may be an electrostatic and/or van der Waals interaction. In some embodiments, the biomolecule may be a polypeptide, DNA or RNA. In some embodiments, the polymer may comprise or may be chitosan, agarose, polylysine, polyethylene glycol (PEG), gelatin or polyvinyl alcohol (PVA). In some embodiments, the biomolecule may be released from the polymer by adding a solvent to the conjugate to form a solution, and changing the temperature of and/or ultrasonicating the solution. In some embodiments, the solution may be incubated according to one of the following conditions: 1) about 50-70° C. for about 5-60 min, about 10-60 min, about 10-15 min, or about 5-15 min, wherein the polymer is chitosan; 2) about 50-70° C. for about 20-60 min, wherein the polymer is agarose; and 3) about 50-70° C. for about 20-60 min, wherein the polymer is polylysine.

In some embodiments, the polymer may be chitosan and the biomolecule may be DNA. In some embodiments, the chitosan to DNA ratio may be about 1.0-156 μg chitosan: about 0.01-50 pmol DNA; about 1.0-156 μg chitosan: about 1.0-10 pmol DNA; about 1.0-100 chitosan: about 1.0-10 pmol DNA; about 13.3-156 μg chitosan: about 1.5 pmol DNA; about 13.3 μg chitosan: about 1.5 pmol DNA; about 97.5 μg chitosan: about 1.5 pmol DNA; about 50 chitosan: about 1.5 pmol DNA; about 20 μg chitosan: about 1.5 pmol DNA; or about 156 μg chitosan: about 1.5 pmol DNA.

In some embodiments, the polymer may be agarose and the biomolecule may be DNA. In some embodiments, the agarose to DNA ratio may be about 1.0-200 μg agarose: about 0.01-50 pmol DNA; about 1.0-200 μg agarose: about 1.0-10 pmol DNA; about 75-150 μg agarose: about 1.2 pmol DNA; or about 75 μg agarose: about 1.2 pmol DNA.

In some embodiments, the polymer may be polylysine and the biomolecule may be DNA. In some embodiments, the polylysine to DNA ratio may be about 0.1-10.0 μg polylysine: about 0.01-50 pmol DNA; about 0.1-10.0 μg polylysine: about 1.0-10 pmol DNA; about 3-10 μg polylysine: about 1.2 pmol DNA; or about 3 μg polylysine: about 1.2 pmol DNA.

In another aspect, the present invention provides a method for controlled release of a biomolecule comprising: a) forming a polymer-biomolecule conjugate by an electrostatic and/or van der Waals interaction; and b) releasing the biomolecule from the polymer by a physical treatment and/or change in an environmental condition.

In some embodiments, the polymer-biomolecule conjugate may be formed by mixing the two components or depositing the two components layer-by-layer before drying. In some embodiments, the temperature for drying may be about 10-95, about 25-80, about 25, about 50, or about 80° C. In some embodiments, the drying time may be about 0.1 h to about 2 month, about 0.1-2 h, about 0.1 h, about 1 h or about 2 h. In some embodiments, the drying condition may comprise a vacuum.

In some embodiments, the releasing of the biomolecule from the polymer may be achieved by adding a solvent to the conjugate to form a solution, and changing the temperature of and/or ultrasonicating the solution. In some embodiments, the solution may be incubated according to one of the following conditions: 1) about 50-70° C. for about 5-60 min, about 10-60 min, about 10-15 min, or about 5-15 min, wherein the polymer is chitosan; 2) about 50-70° C. for about 20-60 min, wherein the polymer is agarose; or 3) about 50-70° C. for about 20-60 min, wherein the polymer is polylysine.

In a further aspect, the present invention provides a solid carrier comprising a biomolecule-polymer conjugate immobilized thereon by a non-covalent interaction, such as an electrostatic and/or van der Waals interaction. In some embodiments, the solid carrier may comprise or may be a chip slide, an ELISA plate, a test tube, or a centrifugal tube. In some embodiments, the material of the solid carrier may be selected from the group consisting of metal, glass, quartz, silicon, porcelain, plastic, rubber and aluminosilicate. In some embodiments, the conjugate may be immobilized by incubating a mixture of the polymer and the biomolecule on the solid carrier under vacuum. In some embodiments, the conjugate may be kept at about 10-95° C. for about 0.1 h to 2 month. In some embodiments, the conjugate may be kept at about 25-80° C. for about 0.1 h to 2 month. In some embodiments, the conjugate may be kept at about 50° C. for about 1 h. In some embodiments, the conjugate may be kept at about 25° C. for about 0.1 h. In some embodiments, the conjugate may be kept at about 80° C. for about 2 h.

In some embodiments, the conjugate may be immobilized by: a) adding a solution comprising the biomolecule on the solid carrier; b) removing the solvent; and c) adding a solution comprising the polymer on the solid carrier under vacuum. In some embodiments, the conjugate may be immobilized by further heating the solid carrier at about 10-95° C. for about 0.1 h to about 2 month. In some embodiments, the conjugate may be immobilized by further heating the solid carrier at about 25-80° C. for about 0.1 h to about 2 month. In some embodiments, the conjugate may be immobilized by further heating the solid carrier at about 50° C. for about 1 h. In some embodiments, the conjugate may be immobilized by further heating the solid carrier at about 25° C. for about 0.1 h. In some embodiments, the conjugate may be immobilized by further heating the solid carrier at about 80° C. for about 2 h. In some embodiments, the solvent may be removed by keeping the chips at about 50° C. for about 1 min under vacuum.

### BRIEF DESCRIPTION OF THE DRAWINGS

FIG. 1 is a schematic representation of the pre-mix mode of the controlled release. 101: the biochip; 102: the biomolecules; 103: the polymers; 104: the reaction solution containing DNA template.

FIG. 2 is a schematic representation of the layer-by-layer mode of the controlled release. 101: the biochip; 102: the biomolecules; 103: the polymers; 104: the reaction solution containing DNA templates.

FIG. 3 shows the data of loop-mediated isothermal amplification with controlled release of the primers in chitosan films. A: the mixture of the primers, templates and buffer; B: controlled release of the primers.

FIG. 4 shows the data of the controlled release test in chitosan films. A: amplification curve with released primers; B: amplification curve without released primers.

FIG. 5 shows the data of loop-mediated isothermal amplification with controlled release of the primers in agarose films.

FIG. 6 shows the data of loop-mediated isothermal amplification with controlled release of the primers in polylysine films.

### DETAILED DESCRIPTION OF THE INVENTION

Described herein is a method for providing dependable immobilization and release of a biomolecule, which is based on a non-covalent conjugation to a polymer.

### A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a”, “an”, and “the” include plural references unless indicated otherwise. For example, “a” dimer includes one or more dimers.

The terms “polynucleotide,” “oligonucleotide,” “nucleic acid” and “nucleic acid molecule” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the term “microfluidic device” generally refers to a device through which materials, particularly fluid borne materials, such as liquids, can be transported, in some embodiments on a micro-scale, and in some embodiments on a nanoscale. Thus, the microfluidic devices described by the presently disclosed subject matter can comprise microscale features, nanoscale features, and combinations thereof.

Accordingly, an exemplary microfluidic device typically comprises structural or functional features dimensioned on the order of a millimeter-scale or less, which are capable of manipulating a fluid at a flow rate on the order of a μL/min or less. Typically, such features include, but are not limited to channels, fluid reservoirs, reaction chambers, mixing chambers, and separation regions. In some examples, the channels include at least one cross-sectional dimension that is in a range of from about 0.1 μm to about 500 μm. The use of dimensions on this order allows the incorporation of a greater number of channels in a smaller area, and utilizes smaller volumes of fluids.

A microfluidic device can exist alone or can be a part of a microfluidic system which, for example and without limitation, can include: pumps for introducing fluids, e.g., samples, reagents, buffers and the like, into the system and/or through the system; detection equipment or systems; data storage systems; and control systems for controlling fluid transport and/or direction within the device, monitoring and controlling environmental conditions to which fluids in the device are subjected, e.g., temperature, current, and the like.

As used herein, the terms “channel,” “micro-channel,” “fluidic channel,” and “microfluidic channel” are used interchangeably and can mean a recess or cavity formed in a material by imparting a pattern from a patterned substrate into a material or by any suitable material removing technique, or can mean a recess or cavity in combination with any suitable fluid-conducting structure mounted in the recess or cavity, such as a tube, capillary, or the like.

As used herein, the terms “flow channel” and “control channel” are used interchangeably and can mean a channel in a microfluidic device in which a material, such as a fluid, e.g., a gas or a liquid, can flow through. More particularly, the term “flow channel” refers to a channel in which a material of interest, e.g., a solvent or a chemical reagent, can flow through. Further, the term “control channel” refers to a flow channel in which a material, such as a fluid, e.g., a gas or a liquid, can flow through in such a way to actuate a valve or pump.

As used herein, “chip” refers to a solid substrate with a plurality of one-, two- or three-dimensional micro structures or micro-scale structures on which certain processes, such as physical, chemical, biological, biophysical or biochemical processes, etc., can be carried out. The micro structures or micro-scale structures such as, channels and wells, electrode elements, electromagnetic elements, are incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips of the present invention can vary considerably, e.g., from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include channels or wells fabricated on the surfaces.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### B. Controlled Release Conjugate

In one aspect, provided herein is a controlled release conjugate comprising a biomolecule conjugated with a polymer through a non-covalent bond, which releases the biomolecule from the polymer by a physical treatment and/or change in an environmental condition.

In some embodiments, the non-covalent bond may be an electrostatic and/or van der Waals interaction. In some embodiments, the biomolecule may be a polypeptide, DNA or RNA. In some embodiments, the polymer may comprise or may be chitosan, agarose, polylysine, polyethylene glycol (PEG), gelatin or polyvinyl alcohol (PVA). In some embodiments, the biomolecule may be released from the polymer by adding a solvent to the conjugate to form a solution, and changing the temperature of and/or ultrasonicating the solution. In some embodiments, the solution may be incubated according to one of the following conditions: 1) about 50-70° C. for about 5-60 min, about 10-60 min, about 10-15 min, or about 5-15 min, wherein the polymer is chitosan; 2) about 50-70° C. for about 20-60 min, wherein the polymer is agarose; and 3) about 50-70° C. for about 20-60 min, wherein the polymer is polylysine.

In some embodiments, the polymer may be chitosan and the biomolecule may be DNA. In some embodiments, the chitosan to DNA ratio may be about 1.0-156 μg chitosan: about 0.01-50 pmol DNA; about 1.0-156 μg chitosan: about 1.0-10 pmol DNA; about 1.0-100 chitosan: about 1.0-10 pmol DNA; about 13.3-156 μg chitosan: about 1.5 pmol DNA; about 13.3 μg chitosan: about 1.5 pmol DNA; about 97.5 μg chitosan: about 1.5 pmol DNA; about 50 μg chitosan: about 1.5 pmol DNA; about 20 μg chitosan: about 1.5 pmol DNA; or about 156 μg chitosan: about 1.5 pmol DNA.

In some embodiments, the polymer may be agarose and the biomolecule may be DNA. In some embodiments, the agarose to DNA ratio may be about 1.0-200 μg agarose: about 0.01-50 pmol DNA; about 1.0-200 μg agarose: about 1.0-10 pmol DNA; about 75-150 μg agarose: about 1.2 pmol DNA; or about 75 μg agarose: about 1.2 pmol DNA.

In some embodiments, the polymer may be polylysine and the biomolecule may be DNA. In some embodiments, the polylysine to DNA ratio may be about 0.1-10.0 μg polylysine: about 0.01-50 pmol DNA; about 0.1-10.0 μg polylysine: about 1.0-10 pmol DNA; about 3-10 μg polylysine: about 1.2 pmol DNA; or about 3 μg polylysine: about 1.2 pmol DNA.

When the polymer is used to immobilize the biomolecules, it can have any suitable structures, e.g., a compact reticular structure or a membrane structure. When the polymer is used to release the biomolecules, it can have a loose reticular structure or a loose membrane structure or a random coil structure.

The conjugate between the polymer and the target biomolecule can be in any suitable form, e.g., being one of the followings: uniform solution, membrane, gel; or one single component that overlaps the other, including particle, membrane and gel. Typically, no special treatment or modification is needed before immobilizing the biomolecules by polymers. Typically, the only step needed is making the relevant solutions of the biomolecule and the polymer.

Any suitable biomolecules can be conjugated. For example, the biomolecule to be conjugated can be an amino acid, a peptide, a protein, a nucleoside, a nucleotide, an oligonucleotide, a nucleic acid, a vitamin, a monosaccharide, an oligosaccharide, a carbohydrate, a lipid and a complex thereof. Chitosan, as an example of the polymers, is a linear polymer, which has abundant amino groups to form electrostatic adherence with many kinds of molecules (including DNA and RNA). It can also form nanoparticle with some biomolecules (Mao, et al. (2001) J. Control. Release 70(3):399-421) to confirm the bonding between them.

In some embodiments, the releasing of the biomolecule from the polymer may be achieved by any suitable methods, e.g., adding a solvent to the conjugate to form a solution, and changing the temperature of and/or ultrasonicating the solution. In some embodiments, the solution may be incubated according to one of the following conditions: 1) about 50-70° C. for about 5-60 min, about 10-60 min, about 10-15 min, or about 5-15 min, wherein the polymer is chitosan; 2) about 50-70° C. for about 20-60 min, wherein the polymer is agarose; or 3) about 50-70° C. for about 20-60 min, wherein the polymer is polylysine.

### C. Methods of Controlled Release of a Biomolecule

In another aspect, the present invention provides a method for controlled release of a biomolecule comprising: a) forming a polymer-biomolecule conjugate by an electrostatic and/or van der Waals interaction; and b) releasing the biomolecule from the polymer by a physical treatment and/or change in an environmental condition.

In some embodiments, the polymer-biomolecule conjugate may be formed by mixing the two components, or depositing the two components layer-by-layer, before drying. In some embodiments, the temperature for drying may be about 10-95, about 25-80, about 25, about 50, or about 80° C. In some embodiments, the drying time may be about 0.1 h to about 2 month, about 0.1-2 h, about 0.1 h, about 1 h or about 2 h. In some embodiments, the drying condition may comprise a vacuum.

In some embodiments, the releasing of the biomolecule from the polymer may be achieved by adding a solvent to the conjugate to form a solution, and changing the temperature of and/or ultrasonicating the solution. In some embodiments, the solution may be incubated according to one of the following conditions: 1) about 50-70° C. for about 5-60 min, about 10-60 min, about 10-15 min, or about 5-15 min, wherein the polymer is chitosan; 2) about 50-70° C. for about 20-60 min, wherein the polymer is agarose; or 3) about 50-70° C. for about 20-60 min, wherein the polymer is polylysine.

In some embodiments, the polymers can be converted to compact or loose form by physical treatment and/or changing the environment conditions. The release of the biomolecules can be controlled by heating or ultrasonicating, which can simplify this releasing method.

The polymers may first be converted to a compact form in one special condition to immobilize the target biomolecules in the biochip. Then the polymers may be converted to a loose form by changing the environment conditions to release the immobilized molecules in the biochip. The whole process may have little influence on the activities of the biomolecules and subsequent reactions, e.g., an amplification reaction.

### D. Solid Carrier

In a further aspect, the present invention provides a solid carrier comprising a biomolecule-polymer conjugate immobilized thereon by a non-covalent interaction, such as an electrostatic and/or van der Waals interaction. In some embodiments, the solid carrier may comprise or may be a chip slide, an ELISA plate, a test tube, or a centrifugal tube. In some embodiments, the material of the solid carrier may be selected from the group consisting of metal, glass, quartz, silicon, porcelain, plastic, rubber and aluminosilicate. In some embodiments, the conjugate may be immobilized by incubating a mixture of the polymer and the biomolecule on the solid carrier under vacuum. In some embodiments, the conjugate may be kept at about 10-95° C. for about 0.1 h to 2 month. In some embodiments, the conjugate may be kept at about 25-80° C. for about 0.1 h to 2 month. In some embodiments, the conjugate may be kept at about 50° C. for about 1 h. In some embodiments, the conjugate may be kept at about 25° C. for about 0.1 h. In some embodiments, the conjugate may be kept at about 80° C. for about 2 h.

In some embodiments, the conjugate may be immobilized by: a) adding a solution comprising the biomolecule on the solid carrier; b) removing the solvent; and c) adding a solution comprising the polymer on the solid carrier under vacuum. In some embodiments, the conjugate may be immobilized by further heating the solid carrier at about 10-95° C. for about 0.1 h to about 2 month. In some embodiments, the conjugate may be immobilized by further heating the solid carrier at about 25-80° C. for about 0.1 h to about 2 month. In some embodiments, the conjugate may be immobilized by further heating the solid carrier at about 50° C. for about 1 h. In some embodiments, the conjugate may be immobilized by further heating the solid carrier at about 25° C. for about 0.1 h. In some embodiments, the conjugate may be immobilized by further heating the solid carrier at about 80° C. for about 2 h. In some embodiments, the solvent may be removed by keeping the chips at about 50° C. for about 1 min under vacuum.

The target biomolecules and polymers can be added into the solid carrier by any suitable methods, e.g., manually (e.g., using a pipette or a capillary) or automatically (e.g., using a spotting machine).

In some embodiments, the implementation procedure of the present invention may comprise: 1) adding a biomolecule and a polymer into a biochip by either mixture or layer-by-layer mode; 2) converting the polymer to a compact form to immobilize the biomolecules; and 3) converting the polymer to a loose form to release the biomolecules after applying a reaction buffer into the biochip. In some embodiments, the released biomolecule can be involved in a further reaction, e.g., an amplification reaction.

The biochip for the target biomolecules (e.g., DNA and RNA) in this invention can be applied to many amplification reactions, including, but not limited to, isothermal amplification (such as LAMP, SDA, NASBA), non-isothermal amplification (such as PCR, LCR). The biochips, which are fabricated according to the methods described in this invention, can be used or commercialized for parallel detections in one chip since the target biomolecules are isolated into different chambers. The preparation methods, materials and equipment for the chip is easy to get at low-cost. Therefore, the methods disclosed herein are of great significance in chip design and manufacturing.

The microfluidic devices of the present invention may comprise a central body structure in which various microfluidic elements are disposed. The body structure includes an exterior portion or surface, as well as an interior portion which defines the various microscale channels and/or chambers of the overall microfluidic device. For example, the body structure of the microfluidic devices of the present invention typically employs a solid or semi-solid substrate that may be planar in structure, i.e., substantially flat or having at least one flat surface. Suitable substrates may be fabricated from any one of a variety of materials, or combinations of materials. Often, the planar substrates are manufactured using solid substrates common in the fields of microfabrication, e.g., silica-based substrates, such as glass, quartz, silicon or polysilicon, as well as other known substrates, i.e., gallium arsenide. In the case of these substrates, common microfabrication techniques, such as photolithographic techniques, wet chemical etching, micromachining, i.e., drilling, milling and the like, may be readily applied in the fabrication of microfluidic devices and substrates. Alternatively, polymeric substrate materials may be used to fabricate the devices of the present invention, including, e.g., polydimethylsiloxanes (PDMS), polymethylmethacrylate (PMMA), polyurethane, polyvinylchloride (PVC), polystyrene, polysulfone, polycarbonate and the like. In the case of such polymeric materials, injection molding or embossing methods may be used to form the substrates having the channel and reservoir geometries as described herein. In such cases, original molds may be fabricated using any of the above described materials and methods.

The channels and chambers of the device are typically fabricated into one surface of a planar substrate, as grooves, wells or depressions in that surface. A second planar substrate, typically prepared from the same or similar material, is overlaid and bound to the first, thereby defining and sealing the channels and/or chambers of the device. Together, the upper surface of the first substrate, and the lower mated surface of the upper substrate, define the interior portion of the device, i.e., defining the channels and chambers of the device. In some embodiments, the upper layer may be reversibly bound to the lower layer.

In the exemplary devices described herein, at least one main channel, also termed an analysis channel, is disposed in the surface of the substrate through which samples are transported and subjected to a particular analysis. Typically, a number of samples are serially transported from their respective sources, and injected into the main channel by placing the sample in a transverse channel that intersects the main channel. This channel is also termed a “sample loading channel.” The sample sources are preferably integrated into the device, e.g., as a plurality of wells disposed within the device and in fluid communication with the sample loading channel, e.g., by an intermediate sample channel.

The systems of the invention may also include sample sources that are external to the body of the device per se, but still in fluid communication with the sample loading channel. In some embodiments, the system may further comprise an inlet and/or an outlet to the micro-channel. In some embodiments, the system may further comprise a delivering means to introduce a sample to the micro-channel. In some embodiments, the system may further comprise an injecting means to introduce a liquid into the micro-channel. Any liquid manipulating equipments, such as pipettes, pumps, etc., may be used as an injecting means to introduce a liquid to the micro-channel.

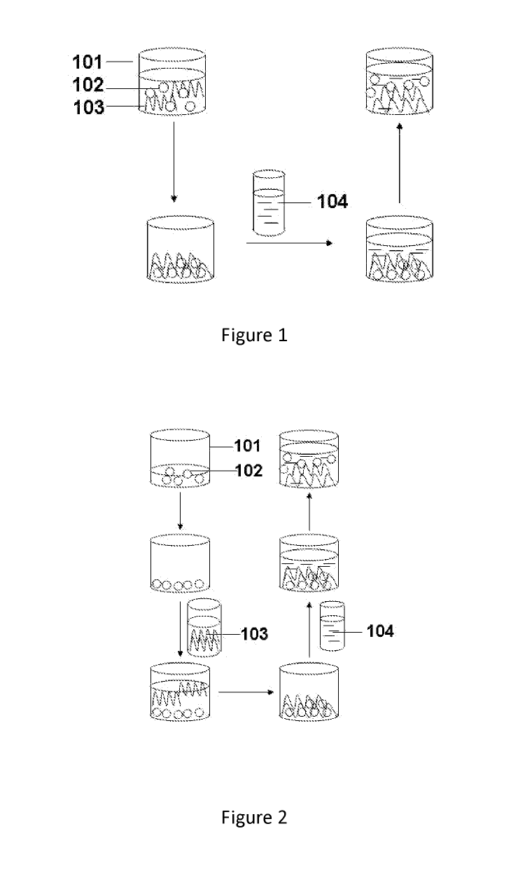

The strategy of the controlled release method with chitosan as an example is shown in FIGS. 1 and 2. In FIG. 1, the solution of the target biomolecules 102 is mixed with the solution of polymer 103 thoroughly. Then the mixture is transferred into the amplification chip 101 before being converted to a compact form, which can immobilize the target biomolecules. The conversion can be achieved by certain physical treatment, including heating, drying and vacuum. After the biomolecules are immobilized, the reaction buffer 104 (containing DNA templates) is added to the chip. The target biomolecules are protected by the polymers to prevent being washed away or cross-contamination during the buffer loading step. In the following amplification reaction, the polymers can be converted to loose form by certain treatment, including heating and humidification. Since the biomolecules are bonded to the polymers non-covalently, they can be released after the conversion of the polymers without any loss of activities. FIG. 2 shows the layer-by-layer mode of the controlled release method. The difference between FIG. 1 and FIG. 2 is that in FIG. 2 the solution of target biomolecules 102 is added into the amplification chip 101 before removing the solvent. The target biomolecules are adsorbed on the chip. Then the polymer solution 103 is added into the chip. The polymer can be converted into a compact form by heating or high vacuum. In this method, the biomolecules and the polymer are applied into the chip layer-by-layer.

### E. Examples

The following examples are offered to illustrate but not to limit the invention.

In all the following examples the biochips were manufactured with PMMA. The only structural requirements for the biochips used in the examples are a reaction chamber for the amplification reaction and a fluidic channel for adding the reaction solution to the reaction chamber. No other structural elements are specifically required.

### Example 1

### Biochip with Chitosan-DNA Conjugates

Chitosan (20° C., 0.5%, 200-500 mPa·s) was purchased from TCI, Japan. DNA sequences were purchased from Sangon, China.

1. Fabrication of the Biochip

Strategy I:

Polymer: chitosan.

Biomolecules: four kinds of single-strand DNA with the following sequences:

A: TTGTAAAACGACGGCCAGTG
B: GACCATGATTACGCCAAGCG
C: GCTTATCGATACCGTCGACCTCGTACGACTCACTATAGGGCGAAT
D: CAGCCCGGGGGATCCACTAGCCTCACTAAAGGGAACAAAAGC

Ratio of the polymer and the biomolecules: chitosan was dissolved in water with the final concentration 0.65% (m/m). Mixture of the four DNA sequences was also prepared, in which the concentration of each sequence was 0.1 μmol/L. Then 15 μL DNA mixture was added to 2.05 μL chitosan solution. In the final solution, the chitosan to each kind of DNA sequence ratio was 13.3 μg: 1.5 pmol. A volume of 0.7 μL final solution that described above was added to each reaction chamber of the biochip.

Immobilization of the biomolecules: the biochip with the chitosan-DNA mixture in reaction chambers was kept in a 50° C. oven for 1 h under high vacuum. After this treatment, the polymer was converted to a compact membrane form that could immobilize the biomolecules efficiently.

Chip storage: the fabricated biochip was sealed by an adhesive film and was kept in room temperature.

Strategy II:

Polymer: chitosan.

Biomolecules: four kinds of single-strand DNA with the following sequences:

A: TTGTAAAACGACGGCCAGTG
B: GACCATGATTACGCCAAGCG
C: GCTTATCGATACCGTCGACCTCGTACGACTCACTATAGGGCGAAT
D: CAGCCCGGGGGATCCACTAGCCTCACTAAAGGGAACAAAAGC

Ratio of the polymer and the biomolecules: chitosan was dissolved in water with the final concentration 0.65% (m/m). Mixture of the four DNA sequences was also prepared, in which the concentration of each sequence was 0.1 μmol/L. Then 0.7 μL DNA mixture was added to each reaction chamber of the biochip. The chip was dried by heating up to 50° C. for 1 min under high vacuum. After depositing the DNA sequences into the reaction chambers, 1.2 μL chitosan solution was also added to each chamber before the chip was dried at 50° C. under high vacuum for 1 h. In each reaction chamber, the chitosan to each kind of DNA sequence ratio was 156 μg: 1.5 pmol.

Immobilization of the biomolecules: the biomolecules were immobilized by the chitosan membrane that covered them with a compact form after the drying.

Chip storage: the fabricated biochip was sealed by an adhesive film and was kept in room temperature.

Strategy III:

Polymer: chitosan.

Biomolecules: four kinds of single-strand DNA with the following sequences:

A: TTGTAAAACGACGGCCAGTG
B: GACCATGATTACGCCAAGCG
C: GCTTATCGATACCGTCGACCTCGTACGACTCACTATAGGGCGAAT
D: CAGCCCGGGGGATCCACTAGCCTCACTAAAGGGAACAAAAGC

Ratio of the polymer and the biomolecules: chitosan was dissolved in water with the final concentration 0.65% (m/m). Mixture of the four DNA sequences was also prepared, in which the concentration of each sequence was 0.1 μmol/L. Then 15 μL DNA mixture was added to different volume of the chitosan solution. In the final solution, the chitosan to each kind of DNA sequence ratio was 97.5 μg: 1.5 pmol; 50 μg: 1.5 pmol; and 20 μg: 1.5 pmol, respectively. A volume of 1.5 μL/0.9 μL/0.85 μL final solution that described above was added to each reaction chamber of the biochip respectively.

Immobilization of the biomolecules: the biomolecules were immobilized by the chitosan membrane that covered them with a compact form after the drying. The drying condition of each sample was different. For the 1.5 μL/0.9 μL/0.85 μL/solution, the drying condition was 25° C. for 0.1 h/80° C. for 2 h/50° C. for 1 h, respectively.

Chip storage: the fabricated biochip was sealed by an adhesive film and was kept in room temperature.

2. The Amplification Reaction Result

Test I: The Activities of the Biomolecules

The biochip was stored after sealing for 3 days before the reaction buffer was added into it. The reaction buffer consisted of template and master mix. The components of the master mix were listed in Table 1.

TABLE 1
The components of the master mix

| | Reactant | Final concentration |
|---|---|---|
| 1 | Bst DNA polymerase | 0.32 U/μL |
| 2 | ThermoPol Reaction Buffer | 1X |
| 3 | dNTPs | 0.4 mmol/L (for each) |
| 4 | EvaGreen dye | 0.6X |
| 5 | BSA | 0.5 mg/mL |
| 6 | betaine | 0.8 mol/L |

The template was purchased from China with the concentration of 10⁵ copies/μL. The ratio of the master mix to template was 23:2 (v/v).

The amplification reaction was carried out at 67° C. for 1 h. A control reaction was also carried out. In the control reaction, the primer, template and the master mix were mixed before the reaction. The condition of the control reaction was 67° C. for 1 h.

The reaction result was detected by real-time fluorescence. The results were compared by the time-of-positive (Tp).

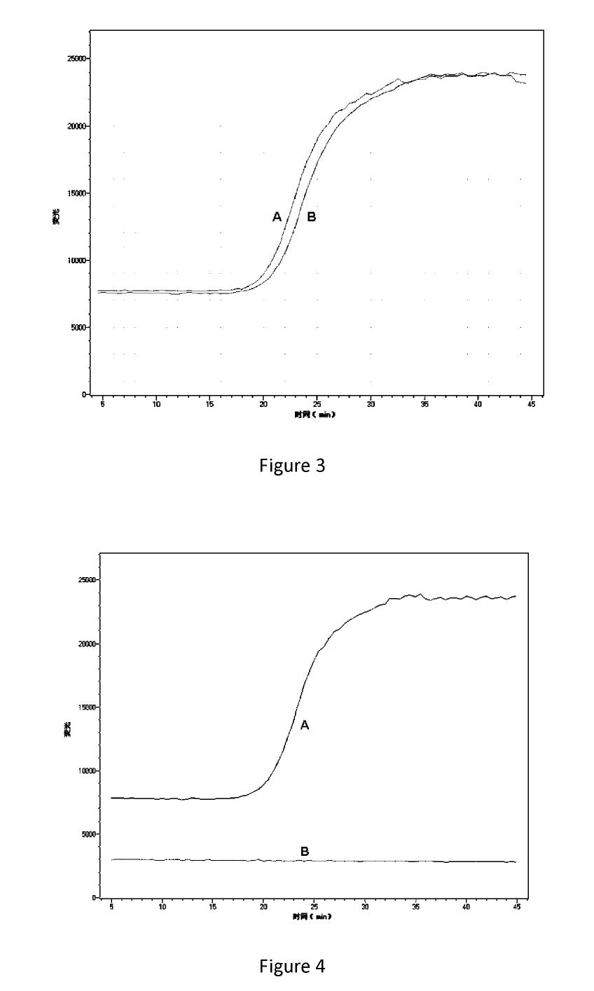

The result of the biochip fabricated by Strategy I was shown in FIG. 3, in which A and B represented the control and test result respectively. It was shown that the shape, fluorescence intensity and the background of the test and control reaction were similar, indicating that efficient reaction was carried out. The Tp of control group and test group was 19 min and 20 min respectively, showing no obvious difference. It was confirmed that the activities of the biomolecules were not destroyed during the controlled release.

The Tp of the biochip fabricated by Strategy II was 22 min.

In the three biochips fabricated by Strategy III, the Tps were 21 min/21 min/20 min respectively. The max difference between test groups was 1 min. The Tp increased with the increase of chitosan concentration, indicating that the higher chitosan concentration was, the slower the biomolecules were released.

Test II: The Controlled Release

The biochips were tested after 3 days of storage.

The biochips were added with water and were kept under different temperature for some time. Since the immobilized DNA-chitosan conjugates were formed as colored thin films, the release of DNA could be observed by microscope. The biochips were observed for 30 min at room temperature, 15 min at 50° C. and 10 min for 70° C. The biomolecules were judged to be released as the edge of the film got blur, while they were judged to be completely released as the solution got colored uniformly.

The result of the biochip fabricated by Strategy I was shown in Table 2.

TABLE 2
The controlled release of biomolecules by chitosan

| Temperature/° C. | Release start/min | Release completed/min |
|---|---|---|
| Room temperature | >20 | >30 |
| 50 | 5 | 15 |
| 70 | 3 | 10 |

The result showed that:

(1) When the water was added into the biochip, the biomolecules were observed to be released after 20 min under room temperature. The biomolecules were not completely released after 30 min, indicating that chitosan could immobilize the molecules efficiently. The fixed molecules had anti-erosion characteristics.

(2) When the water was added into the biochip, the biomolecules were observed to be released after 5 min under 50° C. The biomolecules were completely released after 15 min.

(3) When the water was added into the biochip, the biomolecules were observed to be released after 3 min under 70° C. The biomolecules were completely released after 10 min.

The biomolecules could be released both in case (2) and (3), but the time to complete release of each case was different. The higher the temperature was, the shorter time to complete release was. The whole process of the release could be controlled by changing the temperature.

There was no obvious difference between the results of biochips fabricated by Strategy II/III and the result of the biochip described above.

Test III: Anti-Erosion Characteristics

The biochips were tested after 3 days of storage.

In this test, 7 μL of reaction buffer was added into each chamber of the chip and was taken out after 5 min under room temperature. Then the buffer was heated to 67° C. for 1 h as the condition in amplification reaction, and the real-time fluorescence was also detected. The components in the reaction buffer were the same as described in Test I.

A control reaction was also carried out. In the control reaction, the primer, template and the master mix were mixed before the reaction. The condition of the control reaction was 67° C. for 1 h.

The result of the biochip fabricated by Strategy I was shown in FIG. 4, in which A and B represented the control and test result respectively. No obvious amplification was detected in curve B, indicating that the primers immobilized in the chambers were not washed out during the buffer addition and collection. It was confirmed that the DNA-chitosan film had anti-erosion characteristics, which could prevent the cross-contamination between chambers.

There was no obvious difference between the results of biochips fabricated by Strategy II/III and the result of the biochip described above.

### Example 2

### Biochip with Agarose-DNA Conjugates

The agarose was purchased from Promega. Temperature of solidification: ≦35° C. (4%). Melting point: ≦65° C. (1.5%). Gel strength: ≧500 g/cm².

1. Fabrication of the Biochip

Polymer: agarose.

Biomolecules: four kinds of single-strand DNA with the following sequences:

A: TTGTAAAACGACGGCCAGTG
B: GACCATGATTACGCCAAGCG
C: GCTTATCGATACCGTCGACCTCGTACGACTCACTATAGGGCGAAT
D: CAGCCCGGGGGATCCACTAGCCTCACTAAAGGGAACAAAAGC

Ratio of the polymer and the biomolecules: agarose was dissolved in water with the final concentration 5% (m/m). Mixture of the four DNA sequences was also prepared, in which the concentration of each sequence was 0.1 μmol/L. Then 12 μL DNA mixture was added to different volume of the agarose solution. In the final solution of group 1, the agarose to each kind of DNA sequence ratio was 150 μg: 1.2 pmol. In the final solution of group 2, the agarose to each kind of DNA sequence ratio was 75 μg: 1.2 pmol. A volume of 0.77 μL final solution that described above was added to each reaction chamber of the biochip.

Immobilization of the biomolecules: the biochip with the agarose-DNA mixture in reaction chambers was kept in a 50° C. oven for 1 h under high vacuum. After this treatment, the polymer was converted to a compact membrane form that could immobilize the biomolecules efficiently.

Chip storage: the fabricated biochip was sealed by an adhesive film and was kept in room temperature.

2. The Amplification Reaction Result

The biochips were tested after 3 days of storage.

In this test, 7 μL of reaction buffer was added into each chamber of the chip. The amplification reaction was carried out under 67° C. for 1 h. Four parallel reactions of each group were carried out to detect the repeatability of this method.

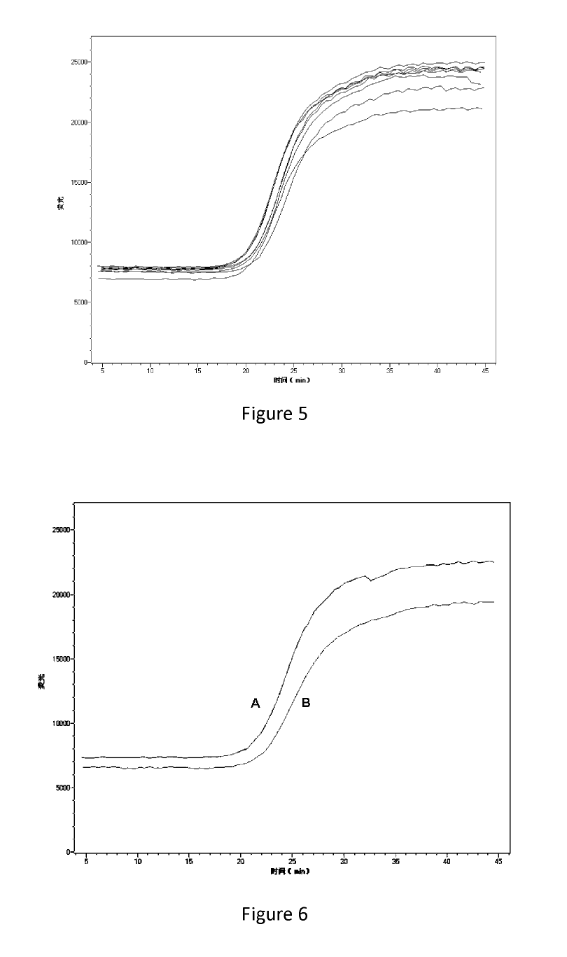

The result of this test was shown in FIG. 5. It was shown that there was no obvious difference between the Tps, which were about 20 min, of the 8 curves. It was confirmed that the activities of the biomolecules were not destroyed during the controlled release. The agarose could be used in a range of concentration with the perfect repeatability.

### Example 3

### Biochip with Polylysine-DNA Conjugates

The polylysine was purchased from Sigma with the code number P9011.

1. Fabrication of the Biochip

Polymer: polylysine with an average molecular weight of 25000-40000 g/mol.

Biomolecules: four kinds of single-strand DNA with the following sequences:

A: TTGTAAAACGACGGCCAGTG
B: GACCATGATTACGCCAAGCG
C: GCTTATCGATACCGTCGACCTCGTACGACTCACTATAGGGCGAAT
D: CAGCCCGGGGGATCCACTAGCCTCACTAAAGGGAACAAAAGC

Ratio of the polymer and the biomolecules: polylysine was dissolved in water with the final concentration 10 mg/mL. Mixture of the four DNA sequences was also prepared, in which the concentration of each sequence was 0.1 μmol/L. Then 12 μL DNA mixture was added to different volume of the polylysine solution. In the final solution of group 1, the polylysine to each kind of DNA sequence ratio was 3 μg: 1.2 pmol. In the final solution of group 2, the agarose to each kind of DNA sequence ratio was 10 μg: 1.2 pmol. A volume of 0.77 μL final solution of group 1 and 0.9 μL of group 2 was added to each reaction chamber of the biochip respectively.

Immobilization of the biomolecules: the biochip with the agarose-DNA mixture in reaction chambers was kept in a 50° C. oven for 1 h under high vacuum. After this treatment, the polymer was converted to a compact membrane form that could immobilize the biomolecules efficiently.

Chip storage: the fabricated biochip was sealed by an adhesive film and was kept in room temperature.

2. The Amplification Reaction Result

The biochips were tested after 3 days of storage.

In this test, 7 μL of reaction buffer was added into each chamber of the chip. The amplification reaction was carried out under 67° C. for 1 h.

The result of this test was shown in FIG. 6, which was similar with the result of Example 2. The Tp of group 1 was 20 min and the Tp of group 2 was 21 min. It was confirmed that the activities of the biomolecules were not destroyed during the controlled release. The polylysine could be used in a range of concentration for the controlled release.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

The advantage of the microvalve described herein includes simple design, controllable operation, broad application range especially for the case that heat effect should not be introduced and the case that closed system should be ensured.

1-52. (canceled)

53. A controlled release conjugate comprising a biomolecule conjugated with a polymer through a non-covalent bond, which releases the biomolecule from the polymer by a physical treatment and/or change in an environmental condition.

54. The conjugate of claim 53, wherein the non-covalent bond is an electrostatic and/or van der Waals interaction.

55. The conjugate of claim 53, wherein the biomolecule is a polypeptide, DNA or RNA.

56. The conjugate of claim 53, wherein the polymer comprises or is chitosan, agarose, polylysine, polyethylene glycol (PEG), gelatin or polyvinyl alcohol (PVA).

57. The conjugate of claim 56, wherein the polymer is chitosan and the biomolecule is DNA, and the chitosan to DNA ratio is about 1.0-156 μg chitosan: about 0.01-50 pmol DNA; about 1.0-156 μg chitosan: about 1.0-10 pmol DNA; about 1.0-100 μg chitosan: about 1.0-10 pmol DNA; about 13.3-156 μg chitosan: about 1.5 pmol DNA; about 13.3 μg chitosan: about 1.5 pmol DNA; about 97.5 μg chitosan: about 1.5 pmol DNA; about 50 μg chitosan: about 1.5 pmol DNA; about 20 μg chitosan: about 1.5 pmol DNA; or about 156 μg chitosan: about 1.5 pmol DNA.

58. The conjugate of claim 56, wherein the polymer is agarose and the biomolecule is DNA, and the agarose to DNA ratio is about 1.0-200 μg agarose: about 0.01-50 pmol DNA; about 1.0-200 μg agarose: about 1.0-10 pmol DNA; about 75-150 μg agarose: about 1.2 pmol DNA; or about 75 μg agarose: about 1.2 pmol DNA.

59. The conjugate of claim 56, wherein the polymer is polylysine and the biomolecule is DNA, and the polylysine to DNA ratio is about 0.1-10.0 μg polylysine: about 0.01-50 pmol DNA; about 0.1-10.0 μg polylysine: about 1.0-10 pmol DNA; about 3-10 μg polylysine: about 1.2 pmol DNA; or about 3 μg polylysine: about 1.2 pmol DNA.

60. The conjugate of claim 53, wherein the biomolecule is released from the polymer by adding a solvent to the conjugate to form a solution, and changing the temperature of and/or ultrasonicating the solution.

61. The conjugate of claim 60, wherein the solution is incubated according to one of the following conditions:
1) about 50-70° C. for about 5-60 min, about 10-60 min, about 10-15 min, or about 5-15 min, wherein the polymer is chitosan;
2) about 50-70° C. for about 20-60 min, wherein the polymer is agarose; or
3) about 50-70° C. for about 20-60 min, wherein the polymer is polylysine.

62. A method for controlled release of a biomolecule comprising:
a) forming a polymer-biomolecule conjugate by an electrostatic and/or van der Waals interaction; and
b) releasing the biomolecule from the polymer by a physical treatment and/or change in an environmental condition.

63. The method of claim 62, wherein the polymer-biomolecule conjugate is formed by mixing the two components or depositing the two components layer-by-layer before drying.

64. The method of claim 63, wherein the temperature for drying is about 10-95° C.; about 25-80° C.; about 25° C.; about 50° C.; or about 80° C., the drying time is about 0.1 h to about 2 month; about 0.1-2 h; about 0.1 h; about 1 h; or about 2 h, and the drying condition comprises a vacuum.

65. The method of claim 62, wherein the releasing of the biomolecule from the polymer is achieved by adding a solvent to the conjugate to form a solution, and changing the temperature of and/or ultrasonicating the solution.

66. The method of claim 65, wherein the solution is incubated according to one of the following conditions:
1) about 50-70° C. for about 5-60 min, about 10-60 min, about 10-15 min, or about 5-15 min, wherein the polymer is chitosan;
2) about 50-70° C. for about 20-60 min, wherein the polymer is agarose; or
3) about 50-70° C. for about 20-60 min, wherein the polymer is polylysine.

67. A solid carrier comprising a biomolecule-polymer conjugate immobilized thereon by a non-covalent interaction, such as an electrostatic and/or van der Waals interaction, using the method of claim 62, wherein the solid carrier comprises or is a chip slide, an ELISA plate, a test tube, or a centrifugal tube, and the material of the solid carrier is selected from the group consisting of metal, glass, quartz, silicon, porcelain, plastic, rubber and aluminosilicate.

68. The solid carrier of claim 67, wherein the conjugate is immobilized by incubating a mixture of the polymer and the biomolecule on the solid carrier under vacuum, and the conjugate is kept at about 10-95° C. for about 0.1 h to 2 month; at about 25-80° C. for about 0.1 h to 2 month; at about 50° C. for about 1 h; at about 25° C. for about 0.1 h; at about 80° C. for about 2 h.

69. The solid carrier of claim 67, wherein the conjugate is immobilized by:
a) adding a solution comprising the biomolecule on the solid carrier;
b) removing the solvent; and
c) adding a solution comprising the polymer on the solid carrier under vacuum.

70. The solid carrier of claim 69, further comprising heating the solid carrier at about 10-95° C. for about 0.1 h to about 2 month; at about 25-80° C. for about 0.1 h to about 2 month; at about 50° C. for about 1 h; at about 25° C. for about 0.1 h; or at about 80° C. for about 2 h.

71. The solid carrier of claim 69, wherein the solvent is removed by keeping the chips at about 50° C. for about 1 min under vacuum.
